(19) United States
(12) Patent Application Publication
(10) Pub. No.: US 2021/0120962 A1
(43) Pub. Date: Apr. 29, 2021

(54) INTELLIGENT BED MONITORING DEVICE AND SYSTEM THEREOF

(71) Applicants: Wen-Hung LIN, Kaohsiung City (TW); Ai. Sleep Co., Ltd., Taipei City (TW)

(72) Inventor: Wen-Hung LIN, Kaohsiung City (TW)

(21) Appl. No.: 16/919,866

(22) Filed: Jul. 2, 2020

Related U.S. Application Data
(60) Provisional application No. 62/927,394, filed on Oct. 29, 2019.

(51) Int. Cl. A47C 20/04; G01J 5/00; G01V 8/20; G01H 17/00; A61B 5/00; A61B 5/0205; A47C 21/00

(52) U.S. Cl. CPC A47C 20/04; G01J 5/0025; G01V 8/20; G01H 17/00; A61B 5/6892; A61B 5/024; A61B 5/0205; A61B 5/0002; A47C 21/003; A61B 2562/0247; A61B 2562/0204; A61B 5/7278

(57) ABSTRACT

An intelligent bed monitoring system and device are provided in this disclosure. The system is applied to a bed frame, and the bed frame includes a bed board. The intelligent bed monitoring system includes a first server, an intelligent bed monitoring device and an electronic device. The intelligent bed monitoring device is communicated with the first server and the electronic device. The intelligent bed monitoring device further includes a pressure sensor, a processor, and a controlling module. The processor is electrically connected with the pressure sensor and the controlling module. The pressure sensor is configured to detect a vibration signal. The processor is configured to generate a physiological information according to the vibration signal, and transmit the physiological information to the first server via a communication interface. The controlling module is configured to control the bed board to adjust the bed board into a plurality of modes.

### CROSS REFERENCE TO RELATED APPLICATION

This application claims the benefit of filing date of U.S. Provisional Application Ser. No. 62/927,394 filed on Oct. 29, 2019. The entirety of said Provisional Application is incorporated herein by reference.

### BACKGROUND OF THE INVENTION

### 1. Field of the Invention

The present invention relates to a monitoring device and system. More particularly, the present application relates to an intelligent bed monitoring device and system thereof.

### 2. Description of Related Art

Generally, everyone needs adequate sleep, however, partial of people may suffer from sleep apnea syndrome, which causes them to have insomnia, daytime naps, fatigue, and cardiac arrhythmia Therefore, in order to improve the user's sleep quality, an intelligent bed monitoring device and monitoring device and system for collecting user's physiological information during sleeping are required.

### SUMMARY OF THE INVENTION

An aspect of the disclosure is to provide an intelligent bed monitoring system. The intelligent bed monitoring system is applied to a bed frame, and the bed frame comprises a bed board, the intelligent bed monitoring system includes a first server, an intelligent bed monitoring device and an electronic device. The intelligent bed monitoring device is communicated with the first server, and the intelligent bed monitoring device includes a pressure sensor, a processor and a controlling module. The processor is electrically connected to the pressure sensor and the controlling module. The pressure sensor is configured to detect a vibration signal. The processor is configured to generate a physiological information according to the vibration signal, and transmit the physiological information to the first server via a communication interface. The controlling module is configured to control the bed board to adjust the bed board into a plurality of modes. The electronic device is configured to transmit a setting signal to the intelligent bed monitoring device, wherein the processor is configured to transmit a first controlling signal to the controlling module according to the physiological information to adjust bed board into one of the plurality of modes.

Another aspect of the disclosure is to provide an intelligent bed monitoring device is applied to a bed frame, and the bed frame comprises a bed board, the intelligent bed monitoring device includes a pressure sensor, a processor, a communication interface and a controlling module. The processor is electrically connected to the pressure sensor, a communication interface and the controlling module. The pressure sensor is configured to detect a vibration signal. The processor is configured to generate a physiological information according to the vibration signal. The communication interface is configured to transmit the physiological information to a first server. The controlling module is configured to control the bed board to adjust the bed board into a plurality of modes, wherein the processor is configured to transmit a first controlling signal to the controlling module according to the physiological information to adjust bed board into one of the plurality of modes.

The foregoing and other objects, advantages, and novel features of the invention will become more apparent from the following detailed description when taken in conjunction with the accompanying drawings.

### BRIEF DESCRIPTION OF THE DRAWINGS

FIG. 1 is a functional block diagram illustrating an intelligent bed monitoring system according to an embodiment of the disclosure.

FIG. 2 is a functional block diagram illustrating an intelligent bed monitoring device according to an embodiment of the disclosure.

FIG. 3 is a schematic diagram of the intelligent bed monitoring device installed on the bed frame according to an embodiment of the disclosure.

FIG. 4A is a schematic diagram of the modes of bed board according to an embodiment of the disclosure.

FIG. 4B is a schematic diagram of the modes of bed board according to an embodiment of the disclosure.

FIG. 4C is a schematic diagram of the modes of bed board according to an embodiment of the disclosure.

### DETAILED DESCRIPTION OF THE PREFERRED EMBODIMENT

It will be understood that, in the description herein and throughout the claims that follow, when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Moreover, “electrically connect” or “connect” can further refer to the interoperation or interaction between two or more elements.

It will be understood that, in the description herein and throughout the claims that follow, although the terms “first,” “second,” etc. may be used to describe various elements, these elements should not be limited by these terms. These terms are only used to distinguish one element from another. For example, a first element could be termed a second element, and, similarly, a second element could be termed a first element, without departing from the scope of the embodiments.

It will be understood that, in the description herein and throughout the claims that follow, the terms “comprise” or “comprising,” “include” or “including,” “have” or “having,” “contain” or “containing” and the like used herein are to be understood to be open-ended, i.e., to mean including but not limited to.

It will be understood that, in the description herein and throughout the claims that follow, the phrase “and/or” includes any and all combinations of one or more of the associated listed items.

It will be understood that, in the description herein and throughout the claims that follow, words indicating direction used in the description of the following embodiments, such as “above,” “below,” “left,” “right,” “front” and “back,” are directions as they relate to the accompanying drawings. Therefore, such words indicating direction are used for illustration and do not limit the present disclosure.

It will be understood that, in the description herein and throughout the claims that follow, unless otherwise defined, all terms (including technical and scientific terms) have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any element in a claim that does not explicitly state “means for” performing a specified function, or “step for” performing a specific function, is not to be interpreted as a “means” or “step” clause as specified in 35 U.S.C. § 112(f). In particular, the use of “step of” in the claims herein is not intended to invoke the provisions of 35 U.S.C. § 112(f).

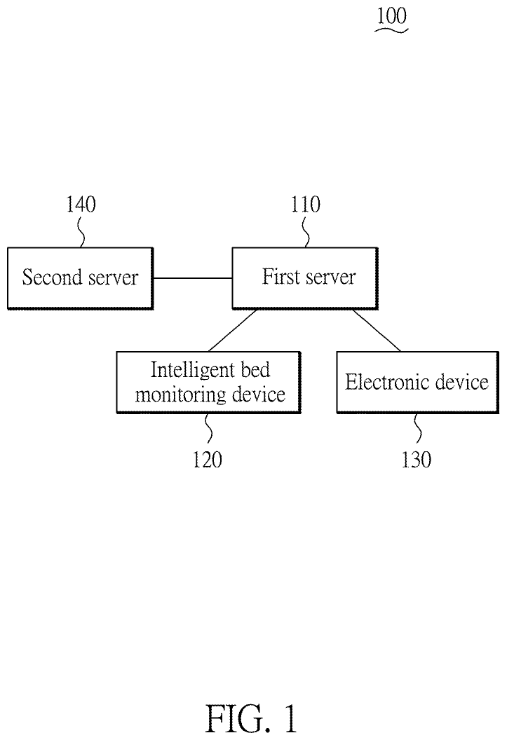

Reference is made to FIG. 1, which is a functional block diagram illustrating an intelligent bed monitoring system 100 according to an embodiment of the disclosure. As shown in FIG. 1, the intelligent bed monitoring system 100 includes a first server 110, an intelligent bed monitoring device 120, an electronic device 130 and a second server 140. The first server 110 is communicated with the intelligent bed monitoring device 120, an electronic device 130 and a second server 140, respectively. The intelligent bed monitoring device 120 is configured to transmit a physiological information to the first server 110, and then the first server 110 is configured to transmit the physiological information to the second server 140. The electronic device 130 is configured to transmit a setting signal to the intelligent bed monitoring device 120.

In the embodiments of the disclosure, the second server 140 can be implemented as a medical cloud server of other business or personal server of user. The electronic device 130 can be implemented as a smart phone, a tablet, or remote control.

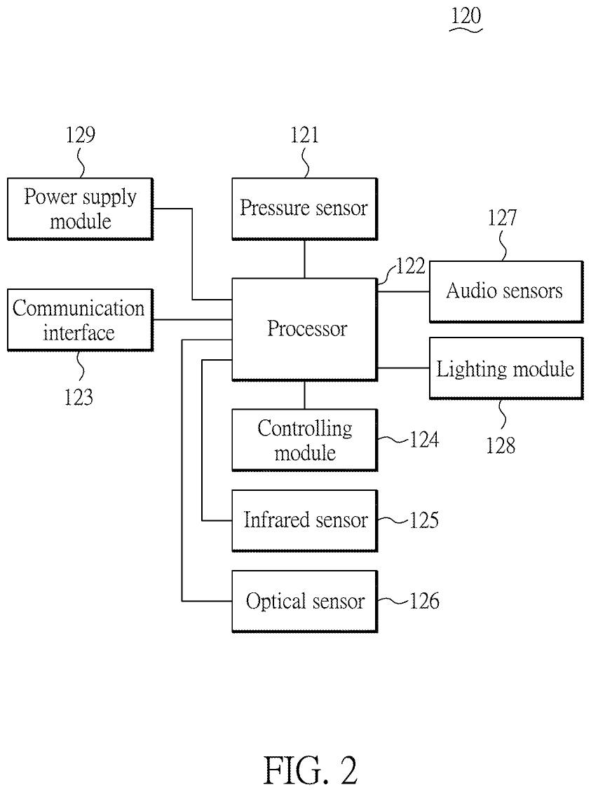

Reference is made to FIG. 2, which is a functional block diagram illustrating an intelligent bed monitoring device 120 according to an embodiment of the disclosure. As shown in FIG. 2, the intelligent bed monitoring device 120 includes a pressure sensor 121, a processor 122, a communication interface 123, a controlling module124, an infrared sensor 125, an optical sensor 126, a plurality of audio sensors 127, a lighting module 128 and a power supply module 129. The processor 122 is electrically connected to the pressure sensor 121, the communication interface 123, the controlling module 124, the infrared sensor 125, the optical sensor 126, the audio sensors 127, the lighting module 128 and the power supply module 129. The processor 122 can be connected to the first server 110 by wire. In another embodiment, the processor 122 can be wirelessly transmitted to the first server 110 through the communication interface 123.

Afterwards, the pressure sensor 121 is configured to detect a vibration signal, and transmit the vibration signal to the processor 122. The infrared sensor 125 is configured to detect an infrared signal, and transmit a first electric signal to the processor 122 according to the infrared signal. The processor 122 is configured to calculate user's temperature according to the first electric signal. Multiple audio sensors 127 are configured to detect multiple audio signals, and transmit the audio signals to the processor 122. The optical sensor 126 is configured to detect a radiation, and transmit the second electric signal to the processor 122 according to the radiation. Then, the processor 122 is configured to transmit a second controlling signal to a lighting module 128 the according to the second electric signal, and enable the lighting module 128.

In the embodiment, the processor 122 can be implemented by a microcontroller, a microprocessor, a digital signal processor, an application specific integrated circuit, a central processing unit, a control circuit and/or a graphics processing unit. The communication interface 123 can be implemented by a global system for mobile communication, a personal handy-phone system, a long term evolution, a worldwide interoperability for microwave access, a wireless fidelity, etc.

Afterwards, the controlling module 124 can be implemented by a quiet motor or other devices that can control the angle of the bed board. The infrared sensor 125 can be implemented by passive infrared sensor or active infrared sensor. The optical sensor 126 can be implemented by a through-beam sensors, diffuse-reflective sensors, retro-reflective sensors or other devices that can detect the object. The audio sensors 127 can be implemented by a microphone or other device which can detect the audio signal. The power supply module 129 can be implemented by battery or the power supply circuit.

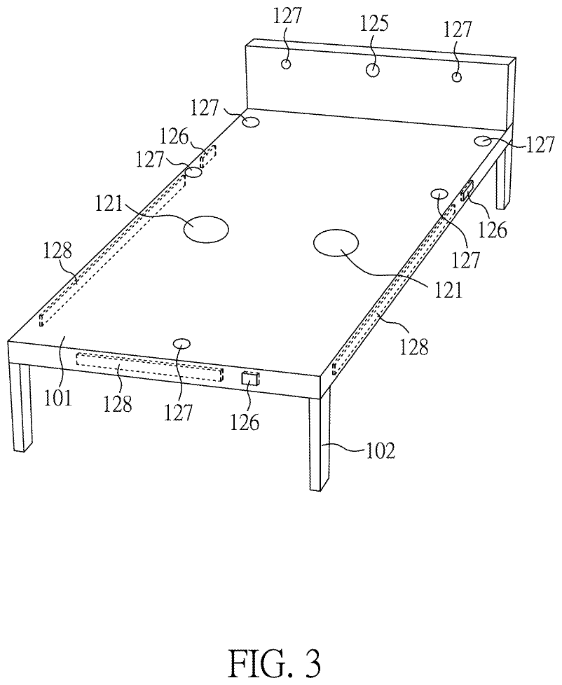

Reference is made to FIG. 3, which is a schematic diagram of the intelligent bed monitoring device 120 installed on the bed frame according to an embodiment of the disclosure. As shown in FIG. 3, the bed body includes the bed board 101 and the bed frame 102. The pressure sensors 121 are installed on the bed board 101, and disposed on the left half and right half of the bed board 101, respectively. It is noticed that, the bed size as shown in FIG. 3 is double size, and thus the pressure sensors 140 are disposed on the bed boards 102 and 103, respectively to detect the physiological signals of different users. In another case, if the bed size is single size, the pressure sensor 140 can be disposed on the center of the bed board.

Afterwards, the infrared sensor 125 is installed on the bed frame 102, and is used to detect the body temperature of liveware on the bed. For example, when the user lies on the mattress (not shown in FIG. 3), the infrared sensor 125 is configured to detect the infrared signal through the face or limbs (the body part exposed outside the comforter), and transmit the first electric signal to the processor 122 according to the infrared signal. Then, the processor 122 is configured to calculate the temperature of the body part exposed outside the comforter according to the first electric signal.

Afterwards, the audio sensors 127 are installed around the bed board 101 to detect user's voice or the ambient audio. For example, the audio sensors 127 are configured to receive user's snoring during sleep, and the processor 122 is configured to determine user's sleep stages (e.g. light sleep, deep sleep and REM sleep).

Afterwards, the optical sensor 126 and the lighting module 128 are installed on the bed frame 102 and located around three sides of bed, so as to detect whether the object is exist. For example, when the user gets up in the middle of the night, the optical sensor 126 located on the side of the bed frame 102 is blocked. In this time, the optical sensor 126 is used to detect the radiation, and transmit a second electric signal to the processor 122 according to the radiation. Then, the processor 122 is configured to enable the lighting module 128 to provide the night lighting according to the second electric signal. In another embodiment, multiple optical sensors 126 are installed on one side of the bed frame to more accurately detect the user's movements.

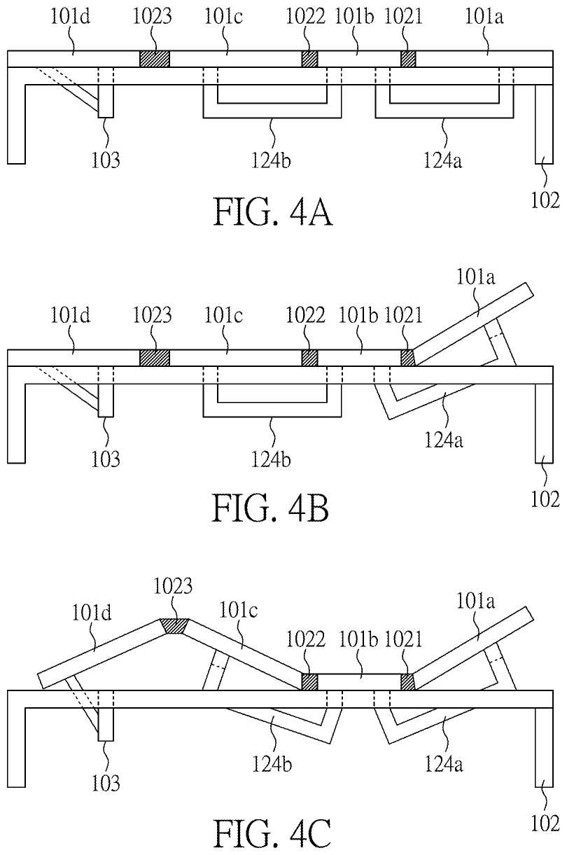

Afterwards, reference is made to FIG. 4A, which is a schematic diagram of the modes of bed board according to an embodiment of the disclosure. As shown in FIG. 4A, the bed board 101 is divided into four blocks, and the bed board 101 includes the blocks 101a, 101b, 101c and 101d. There is a connector 1021 between the blocks 101a and 101b, and the connector 1021 is configured to connect the blocks 101a and 101b. There is a connector 1022 between the blocks 101b and 101c, and the connector 1022 is configured to connect the blocks 101b and 101c. There is a connector 1023 between the blocks 101c and 101d, and the connector 1023 is configured to connect the blocks 101c and 101d. The controlling unit 124a is configured to control the blocks 101a and 101b, and the controlling unit 124b is configured to control the blocks 101c and 101d. The support body 103 is configured to support the block 101d.

Afterwards, reference is made to FIG. 4B and FIG. 4C. FIG. 4B and FIG. 4C are schematic diagram of the modes of bed board according to an embodiment of the disclosure. As shown in FIG. 4B, when the controlling unit 124a receives the first controlling signal transmitted from the processor 122, the controlling unit 124a is configured to upraise the block 101a. It is noticed that, the angle of the block 101a can be adjusted by user. As shown in FIG. 4C, when the controlling unit 124b receives the first controlling signal transmitted from the processor 122, the controlling unit 124b is configured to upraise the block 101c. Then, when the block 101c is upraised, the support body 103 supports the block 101d. It is noticed that, the angle of the block 101c can be adjusted by user.

Afterwards, the processor 122 controls the controlling units 124a and 124b to adjust angle of the blocks 101a and 101c according to the first controlling signal. The user can transmit the setting signal to the processor 122 via the electronic device 130, so that the processor 122 controls the controlling units 124a and 124b to adjust the raising or lowering of the bed board 101.

Afterwards, the bed board 101 can be adjusted into multiple modes. For example, the initial mode can be implemented that the blocks 101a, 101b, 101c and 101d are all in a horizontal state (as shown in FIG. 4A). The first mode can be implemented that the block 101a is in a raised state (as shown in FIG. 4B). The second mode can be implemented that the block 101c is in a raised state (the blocks 101a and 101b are in the horizontal state). The third mode can be implemented that the blocks 101a and 101c are in the raised state (as shown in FIG. 4C). However, the disclosure is not limited thereto.

In the embodiment, the pressure sensor 121 is configured to detect a vibration signal, and transmit the vibration signal to the processor 122. The vibration signal is mainly generated by the physiological information of the user during sleep. For example, the processor 122 analyzes user's heartbeat and breathing through the vibration signal to obtain the physiological signals (e.g. the heartbeat signal and the breathing signal) of the user during sleep.

Afterwards, the pressure sensor 121 also can detect the user's sleep position (e.g. turning over), and thus the processor 122 can automatically adjust the modes of the bed board 101 according to the user's sleep position, the heartbeat information and the breathing information.

Afterwards, the signals detected by the pressure sensor 121, the infrared sensor 125, the optical sensor 126 and the audio sensors 127 are transmitted to the processor 122, and the processor 122 is configured to transmit to the first server via the communication interface 123. Then, the first server 110 is configured to transmit the physiological information related to the user to the second server 140 via the Internet. The second server 140 can be implemented as the medical cloud server of other business, the personal server of user or the server inside the hospital. Therefore, the medical staffs can determine the user's sleep status through the personal physiological information collected by the server.

Based on aforesaid embodiments, the intelligent bed monitoring device and system are capable of collecting physiological data of the user while sleeping; utilizing the pressure sensor, the infrared sensor and the audio sensors to collect user's physiological information. If the user suffers from the cardiac arrhythmia during sleep, the intelligent bed monitoring device can further provide a warning message. In some embodiments, this disclosure is able to achieve the effect of monitoring the sleep status in real-time.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An intelligent bed monitoring system for applying to a bed frame, and the bed frame comprises a bed board, the intelligent bed monitoring system comprising:
a first server;
an intelligent bed monitoring device communicated with the first server, the intelligent bed monitoring device further comprises:
a pressure sensor, configured to detect a vibration signal;
a processor electrically connected to the pressure sensor, is configured to generate a physiological information according to the vibration signal, and transmit the physiological information to the first server via a communication interface; and
a controlling module electrically connected to the processor, is configured to control the bed board to adjust the bed board into a plurality of modes; and
an electronic device communicated with the intelligent bed monitoring device, is configured to transmit a setting signal to the intelligent bed monitoring device;
wherein the processor is configured to transmit a first controlling signal to the controlling module according to the physiological information to adjust bed board into one of the plurality of modes.

2. The intelligent bed monitoring system of claim 1, wherein the intelligent bed monitoring device further comprises:
an infrared sensor electrically connected to the processor, is configured to detect an infrared signal, and transmit a first electric signal to the processor according to the infrared signal.

3. The intelligent bed monitoring system of claim 1, wherein the intelligent bed monitoring device further comprises:
a plurality of audio sensors electrically connected to the processor, are configured to detect a plurality of audio signals.

4. The intelligent bed monitoring system of claim 1, wherein the intelligent bed monitoring device further comprises:
at least one optical sensor electrically connected to the processor, is configured to detect at least one radiation, and transmit a second electric signal to the processor according to the at least one radiation.

5. The intelligent bed monitoring system of claim 4, wherein the processor is configured to transmit a second controlling signal to at least one lighting module the according to the second electric signal, and enable the at least one lighting module.

6. The intelligent bed monitoring system of claim 1, wherein the controlling module further comprises:
a first controlling unit, configured to control a partial of the bed board; and
a second controlling unit, configured to control another partial of the bed board, wherein the processor is configured to control the first controlling unit to adjust the partial of the bed board into a first mode via the first controlling signal; the processor is configured to control the second controlling unit to adjust the another partial of the bed board into a second mode via the first controlling signal.

7. The intelligent bed monitoring system of claim 1, wherein the physiological information comprises a breathing signal and a heartbeat signal.

8. An intelligent bed monitoring device for applying to a bed frame, and the bed frame comprises a bed board, the intelligent bed monitoring device comprising:
a pressure sensor, configured to detect a vibration signal;
a processor electrically connected to the pressure sensor, is configured to generate a physiological information according to the vibration signal;
a communication interface electrically connected to the processor, is configured to transmit the physiological information to a first server; and
a controlling module electrically connected to the processor, is configured to control the bed board to adjust the bed board into a plurality of modes;
wherein, the processor is configured to transmit a first controlling signal to the controlling module according to the physiological information to adjust bed board into one of the plurality of modes.

9. The intelligent bed monitoring device of claim 8, further comprising:
an infrared sensor electrically connected to the processor, is configured to detect an infrared signal, and transmit a first electric signal to the processor according to the infrared signal.

10. The intelligent bed monitoring device of claim 8, further comprising:
a plurality of audio sensors electrically connected to the processor, are configured to detect a plurality of audio signals.

11. The intelligent bed monitoring device of claim 8, further comprising:
at least one optical sensor electrically connected to the processor, is configured to detect at least one radiation, and transmit a second electric signal to the processor according to the at least one radiation.

12. The intelligent bed monitoring device of claim 11, wherein the processor is configured to transmit a second controlling signal to at least one lighting module the according to the second electric signal, and enable the at least one lighting module.

13. The intelligent bed monitoring device of claim 8, wherein the controlling module further comprises:
a first controlling unit, configured to control a partial of the bed board; and
a second controlling unit, configured to control another partial of the bed board, wherein the processor is configured to control the first controlling unit to adjust the partial of the bed board into a first mode via the first controlling signal; the processor is configured to control the second controlling unit to adjust the another partial of the bed board into a second mode via the first controlling signal.

14. The intelligent bed monitoring device of claim 8, wherein the physiological information comprises a breathing signal and a heartbeat signal.
